(19) United States
(12) Patent Application Publication
(10) Pub. No.: US 2010/0286467 A1
(43) Pub. Date: Nov. 11, 2010

(54) DEVICE FOR DRUG DELIVERY AND ASSOCIATED CONNECTIONS THERETO

(76) Inventors: Benny Pesach, Rosh-ha ayin (IL); Gabriel Bitton, Jerusalem (IL); Ron Nagar, Tel Aviv (IL); Ram Weiss, Haifa (IL)

Correspondence Address: MINTZ LEVIN COHN FERRIS GLOVSKY & POPEO, ONE FINANCIAL CENTER, BOSTON, MA, 02111, US

(21) Appl. No.: 12/450,249

(22) PCT Filed: Mar. 19, 2008
(86) PCT No.: PCT/IB2008/051046

§ 371 Date: Jun. 9, 2010

Related U.S. Application Data
(60) Provisional application No. 61/008,274, filed on Dec. 18, 2007.
(60) Provisional application No. 60/970,997, filed on Sep. 10, 2007.
(60) Provisional application No. 60/940,721, filed on May 30, 2007.
(60) Provisional application No. 60/912,698, filed on Apr. 19, 2007.
(60) Provisional application No. 60/895,518, filed on Mar. 19, 2007.
(60) Provisional application No. 60/895,519, filed on Mar. 19, 2007.

(30) Foreign Application Priority Data
Jun. 21, 2007 (US) 11821230

(51) Int. Cl. A61M 5/14; A61M 5/142; A61F 7/00; A61N 7/00; A61N 5/00; A61N 1/00; A61N 2/00

(52) U.S. Cl. 600/9; 604/257; 604/151; 604/114; 604/22; 604/21; 604/508

(57) ABSTRACT

Embodiments of the present invention are directed to systems, apparatuses, devices and methods for improving, modifying and/or stabilizing pharmacokinetic and/or pharmacodynamic profile of a drug infused into the tissue by a catheter and absorbed into the blood or lymphatic system. In some embodiments, infusion sets provide fluid and electrical connection between a catheter assembly/treatment element and at least one of a fluid source and a second source.

### CROSS-REFERENCE TO RELATED APPLICATIONS

The present application claims priority to U.S. Provisional Patent Application Ser. No. 60/895,518, filed Mar. 19, 2007, U.S. Provisional Patent Application Ser. No. 60/895,519, filed Mar. 19, 2007, U.S. Provisional Patent Application Ser. No. 60/912,698, filed Apr. 19, 2007, U.S. Provisional Patent Application Ser. No. 60/940,721, filed May 30, 2007, U.S. patent application Ser. No. 11/821,230, filed Jun. 21, 2007, U.S. Provisional Patent Application No. 60/970,997, filed Sep. 10, 2007, and entitled “Method and Device for Drug Delivery” and to U.S. Provisional Patent Application No. 61/008,274, filed Dec. 18, 2007, and entitled “Device for Drug Delivery and Associated Connections Thereto”. The disclosures of the above applications are incorporated herein by reference in their entireties.

### BACKGROUND OF THE INVENTION

1. Field of the Invention

The present invention relates to systems, apparatuses, devices and methods for delivering drugs to a patient. In particular, the present invention relates to systems, apparatuses, devices and methods for subcutaneous infusion of drugs or substances and using energy sources to improve effectiveness of the infused drugs and more specifically, to devices which enable connection between elements of a drug delivery system.

2. Background of the Invention

Diabetes is a very serious illness affecting millions of people today. Many diabetic patients require injection of insulin to maintain proper levels of glucose in their blood in order to survive. Such injections of insulin are done using drug delivery systems.

Many medical treatment systems and methods involve drug delivery systems that employ subcutaneous infusions of therapeutic fluids, drugs, proteins, and other compounds. Such delivery systems and methods, especially in the area of insulin delivery, have made use of subcutaneous catheters and continuous subcutaneous insulin infusion (“CSII”) pumps. In conventional insulin pumps, the pump is configured to be attached to a disposable thin plastic tube or a catheter through which insulin passes into the tissue. The catheter can be inserted transcutaneously, typically through the skin of the patient's abdomen, and is changed every two to three days. New types of insulin pumps, such as the OmniPod pump manufactured by Insulet Corporation, do not have an external catheter and, instead, a catheter port is embedded into the pump mechanism.

In many instances, patients require insulin delivery around the clock to keep proper levels of glucose in their blood. Insulin can be delivered at a basal rate or in bolus doses. The basal rate represents insulin that is continuously delivered to the patient. Such a continuous delivery of insulin keeps the blood glucose level in the desired range between meals and overnight. The bolus dose is an amount of insulin delivered to the patient according to food intake at meals, particularly carbohydrates. When the patient consumes food, his or her levels of glucose rise. Some conventional pump mechanisms are configured to react upon command, or by way of an algorithm, to the increase in glucose levels by delivering a bolus dose of insulin that matches the rise in the level of glucose and prevents large fluctuations in glucose levels. However, this attempt at control is confounded by the fact that there is usually a variable profile of the absorption of insulin from the injection site to the blood circulation. This variability of the insulin absorption results in an error of up to 30% in insulin levels in the blood and hence results in variability of the insulin effect. Such variability, in turn, causes extreme variability in the resulting glucose levels, which may cause hyperglycemic and hypoglycemic events. In any case, such variability itself has been shown to be potentially damaging to organs and body systems. This is discussed in Lutz Heinemann, “Variability of Insulin Absorption and Insulin Action”, Diabetes Technology & Therapeutics, Vol. 4 No. 5, 2002.

### SUMMARY OF THE INVENTION

The present invention relates to devices for improving, modifying and/or stabilizing pharmacokinetic and/or pharmacodynamic profiles of a drug infused into the tissue by a catheter and absorbed into the blood or lymphatic system. The devices disclosed in some embodiments of the present application apply additional treatment or stimulation to the vicinity of the drug delivery site. The treatment(s) may feature one or more of the tissue treatment treatments modalities, as disclosed in co-owned, co-pending U.S. patent application Ser. No. 11/812,230 and U.S. Provisional Patent Application Ser. Nos. 60/895,518, 60/895,519, 60/912,698, and 60/940,721 (hereinafter referred to as “Commonly Owned Applications”), the disclosures of which are incorporated by reference herein in their entireties. As stated in the Commonly Owned Applications, such treatments may include, but are not limited to, heating, modifying temperature, massaging, mechanical vibration, acoustic vibration, ultrasound, suction, infusion of an additional substance or chemical, applying a low electric field, applying a low magnetic field, light irradiation, radiofrequency (“RF”) irradiation, microwave (“MW”) irradiation, etc.

According to some embodiments of the present invention, the devices may include a catheter for insertion within the tissue to infuse a substance into the infused tissue region. The infused tissue region (also referred to as “the infused region”) can be one of the layers of the skin, the subcutaneous tissue, deeper tissue elements within any organ, or viscera. Additionally, the catheter or infusion set can have a securing mechanical part that adheres to the skin and secures the catheter to its location and prevents it from being pulled out accidentally. The proximal end of the catheter is connected to a drug delivery device which controls the infusion profile of the drug, which can be a pump. In some embodiments, the drug delivery device also controls the additional treatment applied to the infused tissue region and/or provides electrical power to it. In such embodiments, electrical wires connect the drug delivery device and the treatment device located in the catheter and/or the catheter securing element.

In some embodiments, the wires connecting the treatment device and the drug delivery pump can be embedded in the catheter tube that is connected to the pump or attached to the outer side of the tube or disposed on a cable attached to the tube. Embedding or attaching the wires to the tube enables the device to be more comfortable for the user (e.g., when it is being worn, handled, etc.). The wires can be connected to the catheter unit that includes the treatment element. However, in many cases, e.g., in the insulin infusion sets, the catheter has a connector that allows the tube to be disconnected from the catheter when needed, for instance when taking a shower. In such cases, the electrical wires can be disconnected as well in a comfortable way. In some embodiments, the present invention provides several exemplary configurations that ease connection and disconnection processes for the user of the device, by having the tube connector and the electrical wires connector disposed in the same housing. Other configurations are possible as well.

In some embodiments, any one or more connectors, connector assemblies and the like may be capable of repeated connection and disconnection, or may be arranged such that the connector(s) is only capable of one time connection and/or disconnection.

In some embodiments, the connector housing includes a clip, flexible element and/or locking mechanism that enables disconnection of the connector only when the locking mechanism is pressed or opened. Such a locking mechanism also reduces the chance of leakage of the infusion fluid from the connector and secures the electrical connection of the wires to the treatment element.

In some embodiments, a plurality of electrical wires, for example three electrical wires, can be used for controlling the treatment device by the pump unit and for connecting a sensor (for example) that measures the treatment level or effect in order to stabilize the treatment effect to the required level. In other cases of treatments, sensors and device configurations, a different number of wires may be connected through the connector. Additionally, to illustrate the present invention's operation, an exemplary treatment method of heating the drug infused tissue is chosen to demonstrate the effect, but the following embodiments and concepts and methods for connecting the infusion sets and electrical wires can be used for any other treatments disclosed in the Commonly Owned Applications. Such treatment methods include, but are not limited to, one or more of the following: heating, cooling, intermittent temperature change, mechanical vibration, acoustic vibration, massaging, ultrasound, suction, electric current, magnetic field, electric field, optical energy, radio frequency irradiation, microwave irradiation, or the like.

In some embodiments, the electrical wire contacts may be placed on the connector part that is also attached to the catheter unit. Such contacts can be optionally covered with a cover when the connector is disconnected. This cover may be useful in different situations, for example, when taking a shower. In some embodiments, covering the electrical wires contacts can be performed manually by the user, for example, by placing a cover that fits over the connector housing and covers the electrical wire contacts and/or the catheter tube. In some embodiments, the cover may be integrated into the electrical wire contacts, thus, allowing the electrical wire contacts to be covered automatically when connector is being disconnected from its housing.

In some embodiments of the invention, any one or more of the electrical contacts disclosed herein, as associated with any component or provided unilaterally, may also comprise an electrical connector, an electrical conductor and/or any other means by which electrical conductivity or communication can be obtained. It is also worth noting, that in some embodiments, “electrical wire” may be used interchangeably with the phrase “electrical conductor”.

In some embodiments, such as those disclosed in the Commonly Owned Applications and/or the present application, the catheter unit can include at least one electronic component. In some embodiments, an optional function of the at least one or more electronic components provides an electronic unique identifier for example including a serial number associated with the infusion set being used, such as the one provided by Maxim DS2433 1-Wire EEPROM. The optional stored serial number can be read electronically by the drug delivery device and/or by a third, auxiliary and/or peripheral unit that may be attached to the drug delivery pump.

When reading and identifying a particular serial number, the processing unit controlling the drug delivery process, for example, and/or the tissue treatment element, can also identify the time, for example, of the onset of treatment protocol. In some embodiments, timing the tissue treatment element with an internal clock can therefore determine how long a specific drug infusion set is used. The processing unit may limit the use of a specific infusion set to a preset time period according to the manufacturer guidelines, such as, three days. For a situation in which a specific infusion set is used beyond such predetermined time period, the processing unit can alert the user and/or disable the treatment operation and/or disable the drug delivery operation and/or perform another act to induce or compel the user (or another individual) to replace the infusion set.

In some embodiments, the serial number of the infusion set may be further used to obtain data relevant to a specific infusion set model or infusion set manufacturing data. For example, such data includes, but is not limited to, calibration data for the treatment device and/or sensors, such as thermistors or the like. In some embodiments, the specific calibration data or other infusion set and treatment information are stored at an electronic component, such as a Maxim DS2433 1-Wire EPROM (as a non-limiting example), disposed in the infusion set. In some embodiments, the serial number can be similar for a specific manufacturing lot. In some embodiments, the serial number can be similar for a specific model. In some embodiments, the serial number includes an infusion set unique number and/or manufacturing lot and/or model information. In some embodiments, the serial number and attribute information of the infusion set can also used by the processing unit for documentation.

In some embodiments, the electronic information component is disposed in the catheter part of the infusion set and is connected to the processing unit that is disposed in the drug delivery device and/or a second unit (may also be referred to as third unit, fourth unit, auxiliary unit, and the like) through the wires and/or connectors, as discussed above. In some embodiments, additional wires may be required between the catheter drug delivery unit and the processing unit for communication and/or power supply. In some embodiments, the same wires may be used. In some embodiments, the infusion set electronic information component is disposed in a detachable part of the infusion set and connected to the processing unit disposed in the drug delivery device and/or the third unit through the wires described in the present invention.

In some embodiments, the infusion set electronic information component is disposed in the connector at the other side of the infusion set, close to the drug delivery device and/or a secondary/third/auxiliary unit through a connector that combines the infusion set tube and/or electric wires, as disclosed in the Commonly Owned Applications.

In some embodiments, as disclosed in the Commonly Owned Applications, the energy source, such as batteries for the treatment element, can be disposed in the disposable infusion set. In some embodiments, the processing unit can use specific information from the infusion set to limit the use of the disposable infusion set to a specific preset time period, based on the manufacturer guidelines, to a number of operations of the treatment element, to a period of usage of the treatment element, and/or to a threshold of a certain percentage of the battery power to prevent malfunction of the treatment element because of empty batteries. In case an infusion set is used beyond such period, the processing unit may alert the user, disable the treatment operation, and/or disable the drug delivery operation and/or other operations to induce the user to replace infusion set with its disposable batteries.

In some embodiments, the specific information of the infusion set is implemented by optical information, such as a bar code marked on one of the parts of the infusion set or on the infusion set package. In this case, the processing unit has an optical processing mechanism such as bar code reader to read the infusion set specific information. Once the user replaces the infusion set, the optical marking can be presented to the sensor, such that the processing unit receives the new infusion set related information and uses it, as discussed above.

In some embodiments, the specific information and identification for the infusion set may be implemented by Radio Frequency Identification (“RFID”), such as a small RFID chip attached to one of the parts of the infusion set or on the infusion set package. In this case, the processing unit has an RF mechanism such as RFID reader to read the infusion set specific information. Once the user replaces the infusion set, the processing unit can receive the new infusion set related information through RF communication and uses it, as discussed above.

In some embodiments of drug delivery devices that include a reusable part, such as a drug delivery pump, and a disposable part, such as an infusion set, the disposable part may include specific information that can be used by one of the methods discussed above. Such information includes information about an electronic component, an RFID, or optical means attached to one of the components of the disposable part or its package. In some embodiments, the reusable part's processing unit obtains specific information about the disposable part once replaced, and then uses that information to limit the usage of the disposable part to predetermined time period according to the manufacturer or other guidelines.

In some embodiments for insulin delivery in which the infusion set is disposable, the infusion set can include specific information, used by one of the methods discussed above, through a device attached to one of the components of the infusion set or its package, such that the insulin pump's processing unit obtains the specific information about the infusion set once the user replaces the infusion set, and then uses that information to limit the usage of the infusion set to a predetermined time period, such as three days, according to the manufacturer or other guidelines.

In some embodiments of the invention, an infusion set for delivering a therapeutic fluid to a patient is provided and may include a treatment element capable of providing a treatment adjacent a catheter, at least one first electrical contact in electrical communication with the treatment element and a catheter assembly. The catheter assembly may include a catheter housing, a catheter capable of insertion transcutaneously into a patient, where the catheter is in fluid communication with the fluid inlet port, and a fluid inlet port in fluid communication with the catheter. The infusion set may also include a catheter connector assembly capable of connection with the catheter assembly, the connector assembly. The catheter connecting assembly may include a catheter connector housing, a fluid delivery tube capable of communicating therapeutic fluid from a fluid unit to the catheter upon connection of the catheter connector assembly with the catheter assembly, at least one second electrical contact, and at least one electrical wire in electrical communication with the at least one second electrical contact. The infusion set may also include a fluid unit connector assembly capable of connection with at least the fluid unit, where the unit connector assembly may include a unit connector housing, a fluid receiving port capable of receiving therapeutic fluid from the fluid unit upon connection of the unit connector assembly with the fluid unit, and at least one third electrical contact in communication with the at least one electrical wire capable of allowing electrical communication a fourth electrical contact provided on at least one of the fluid unit and a second unit. The infusion set may further include a securing member for securing at least one of the treatment element, catheter assembly and catheter connector assembly to the patient. The securing member may be an adhesive or other means (e.g., belt, clip and the like).

In some embodiments of the invention, a catheter for delivering a therapeutic fluid to a patient is provided and may include a treatment element capable of providing a treatment adjacent a catheter, at least one first electrical contact and a catheter assembly. The catheter assembly may include a catheter housing, a catheter capable of insertion transcutaneously into a patient, wherein the catheter is in fluid communication with a fluid inlet port, and a fluid inlet port in fluid communication with the catheter. The catheter may also include a catheter connector assembly capable of connection with the catheter assembly, where the connector assembly may include a catheter connector housing, a fluid delivery tube capable of communicating therapeutic fluid from a fluid unit to the catheter via the fluid inlet port upon connection of the catheter connector assembly with the catheter assembly, at least one second electrical contact, and at least one electrical wire in electrical communication with the at least one second electrical contact.

In some embodiments of the invention a catheter connector assembly for connection with a catheter assembly capable of delivering a therapeutic fluid to a patient is provided and may include a catheter connector housing, a fluid delivery tube capable of communicating therapeutic fluid from a fluid unit to a catheter positioned within the catheter housing upon connection of the catheter connector assembly with a catheter assembly, at least one electrical contact, and at least one electrical wire in electrical communication with the at least one electrical contact.

In some embodiments of the present invention, a catheter fluid unit connector assembly for connection with a fluid dispensing unit capable of delivering a therapeutic fluid to a patient is provided and may include a fluid unit connector housing, a fluid receiving port capable of receiving therapeutic fluid from a fluid unit upon connection of the unit connector assembly with the fluid unit, and at least one first electrical contact in communication with the at least one electrical wire capable of allowing electrical communication a second electrical contact provided on at least one of the fluid unit and a second unit.

In some embodiments of the present invention, a catheter fluid unit connector assembly for connection with a fluid dispensing unit capable of delivering a therapeutic fluid to a patient is provided and may include a fluid unit connector housing, a fluid receiving port capable of receiving therapeutic fluid from a fluid unit upon connection of the unit connector assembly with the fluid unit, and an electronic element with at least one first electrical contact in communication with the at least one electrical wire capable of allowing electrical communication a second electrical contact provided on at least one of the fluid unit and a second unit.

In some embodiments of the present invention, a therapeutic fluid delivery system for delivering a therapeutic fluid to a patient is provided and may include a fluid unit and an infusion set according to embodiments described in the present disclosure (e.g., previous summarized embodiments noted above).

In some embodiments of the invention, an infusion set for delivering a therapeutic fluid to a patient is provided and may include a treatment element capable of providing a treatment adjacent a catheter, where a first side of the treatment element includes at least one first electrical contact, a catheter assembly including a catheter housing, a catheter capable of insertion transcutaneously into a patient, wherein the catheter is in fluid communication with a fluid inlet port, and a fluid inlet port in fluid communication with the catheter. Such embodiments may also include a catheter connector assembly capable of connection with the catheter assembly, where the connector assembly may include a catheter connector housing, a fluid delivery tube capable of communicating therapeutic fluid from a fluid unit to the catheter upon connection of the catheter connector assembly with the catheter assembly, at least one second electrical contact, and at least one electrical wire in electrical communication with the at least one second electrical contact. The infusion set may also include a fluid unit connector assembly capable of connection with at least the fluid unit, where the unit connector assembly may include a fluid unit connector housing, a fluid receiving port capable of receiving therapeutic fluid from the fluid unit upon connection of the unit connector assembly with the fluid unit, and at least one third electrical contact in communication with the at least one electrical wire capable of allowing electrical communication a fourth electrical contact provided on at least one of the fluid unit and a second unit.

In some embodiments of the present invention, an infusion set for delivering a therapeutic fluid to a patient is provided and may include a treatment element capable of providing a treatment adjacent a catheter, where a first side of the treatment element includes at least one first electrical contact, a catheter assembly including a catheter housing and a catheter capable of insertion transcutaneously into a patient. The catheter is in fluid communication with a fluid inlet port. The catheter assembly may also include a fluid inlet port in fluid communication with the catheter and at least one electrical wire in electrical communication with the at least one first electrical contact. The infusion set may also include a fluid unit connector assembly capable of connection with at least the fluid unit, where the unit connector assembly may include a unit connector housing, a fluid receiving port capable of receiving therapeutic fluid from the fluid unit upon connection of the unit connector assembly with the fluid unit, and at least one second electrical contact in communication with the at least one electrical wire capable of allowing electrical communication a third electrical contact provided on at least one of the fluid unit and a second unit.

In some embodiments of the present invention, an infusion set for delivering a therapeutic fluid to a patient is provided and may include a treatment element capable of providing a treatment adjacent a catheter, at least one first electrical contact in electrical communication with the treatment element, and a catheter assembly including a catheter housing, a catheter capable of insertion transcutaneously into a patient, where the catheter is in fluid communication with a fluid inlet port, a fluid inlet port in fluid communication with the catheter and a catheter connector assembly capable of connection with the catheter assembly. The connector assembly may include a catheter connector housing, a fluid delivery tube capable of communicating therapeutic fluid from a fluid unit to the catheter upon connection of the catheter connector assembly with the catheter assembly, at least one second electrical contact, and at least one electrical wire in electrical communication with the at least one second electrical contact.

In some embodiments of the present invention, an infusion set for delivering a therapeutic fluid to a patient is provided and may include a catheter assembly having a catheter housing, a catheter capable of insertion transcutaneously into a patient, wherein the catheter is in fluid communication with a fluid inlet port, a fluid inlet port in fluid communication with the catheter, an electronic element includes at least one first electrical contact and a catheter connector assembly capable of connection with the catheter assembly. The connector assembly may include a catheter connector housing, a fluid delivery tube capable of communicating therapeutic fluid from a fluid unit to the catheter upon connection of the catheter connector assembly with the catheter assembly, at least one first electrical contact, and at least one electrical wire in electrical communication with the at least one first electrical contact. The infusion set may also include a fluid unit connector assembly capable of connection with at least the fluid unit, where the unit connector assembly includes a fluid unit connector housing, a fluid receiving port capable of receiving therapeutic fluid from the fluid unit upon connection of the unit connector assembly with the fluid unit, and at least one third electrical contact in communication with the at least one electrical wire capable of allowing electrical communication a fourth electrical contact provided on at least one of the fluid unit and a second unit.

Some embodiments of the present invention provide for an infusion set for delivering a therapeutic fluid to a patient which may include a catheter assembly having a catheter housing, a catheter capable of insertion transcutaneously into a patient, wherein the catheter is in fluid communication with a fluid inlet port, a fluid inlet port in fluid communication with the catheter, an electronic element and at least one first electrical contact. The infusion set may also include a catheter connector assembly capable of connection with the catheter assembly, where the connector assembly includes a catheter connector housing, a fluid delivery tube capable of communicating therapeutic fluid from a fluid unit to the catheter upon connection of the catheter connector assembly with the catheter assembly, at least one second electrical contact, and at least one electrical wire in electrical communication with the at least one first electrical contact.

Some embodiments of the present invention provide for an infusion set for delivering a therapeutic fluid to a patient and may include a catheter assembly having a catheter housing, a catheter capable of insertion transcutaneously into a patient, wherein the catheter is in fluid communication with the fluid inlet port, a fluid inlet port in fluid communication with the catheter, an electronic element; and at least one first electrical contact. The infusion set may also include a catheter connector assembly capable of connection with the catheter assembly, the connector assembly having a catheter connector housing, a fluid delivery tube capable of communicating therapeutic fluid from a fluid unit to the catheter upon connection of the catheter connector assembly with the catheter assembly, at least one second electrical contact, and at least one electrical wire in electrical communication with the at least one first electrical contact. The infusion set may further include a fluid unit connector assembly capable of connection with at least the fluid unit, the fluid unit connector assembly including a unit connector housing, a fluid receiving port capable of receiving therapeutic fluid from the fluid unit upon connection of the unit connector assembly with the fluid unit, and at least one third electrical contact in communication with the at least one electrical wire capable of allowing electrical communication a fourth electrical contact provided on at least one of the fluid unit and a second unit.

In some embodiments of the present invention, an infusion set for delivering a therapeutic fluid to a patient comprising a catheter assembly is provided and may include a catheter housing, a catheter capable of insertion transcutaneously into a patient, wherein the catheter is in fluid communication with a fluid inlet port, a fluid inlet port in fluid communication with the catheter, and a securing member for securing at least one of the treatment element, catheter assembly and catheter connector assembly to the patient.

The above noted embodiments may, where applicable, include the following additional features:
one or more connectors are capable of removable connection and/or reconnection;
at least one connector assembly is connectable with a corresponding receiving area using a coupling mechanism selected from the group consisting of: a snap lock, clip lock, hook and loop, male and female, pressure lock, a twist lock and any combination of the foregoing;
one or more electrical contacts comprise a connector;
a movable or removable cover (which may be manually removable/movable, or may be automatic) provided with the catheter housing capable of covering at least one electrical contact and a fluid inlet port at least prior to use, where the cover may be rotatable, such that rotating the cover exposes at least one of the electrical contact and a fluid inlet port;
the catheter connection assembly connects to the catheter assembly upon rotation;
rotation of the catheter connector assembly in a first direction upon connection of the catheter connector assembly with the catheter assembly enables operation of at least one of the first unit and the second unit;
rotation of the catheter connector assembly in a second direction upon disconnection of the catheter connector assembly from the catheter assembly disables operation of at least one of the first unit and the second unit;
connection of at least one of the catheter connector assembly and the fluid unit connector assembly enable operation of at least one of the fluid unit and the second unit;
a locking member for locking the catheter connector assembly to the catheter assembly;
a locking member comprising at least one flexible portion provided on the catheter connector assembly received by a corresponding receiving portion in the catheter assembly, where the flexible portion is movable via external pressure applied to the flexible clip, such that application of such pressure unlocks the catheter connector assembly from the catheter assembly;
a locking member for locking the fluid unit connector assembly to at least the fluid unit;
at least one sensor;
the second unit comprises at least one of a power unit separate and apart from the fluid unit, a controller that controls at least one of the treatment element, operation and/or control of the fluid unit and/or the second unit, a user interface for initiating and/or monitoring treatment, an indicator for indicating the treatment status, a sensor that detects fluid dispensing by first unit, and an adaptor to attach second unit to first unit;
a micro-processor;
the second unit further comprises a sensor for sensing at least activation of the first unit;
the catheter assembly further comprises a plurality of first electrical contacts, the catheter connector assembly further comprises a plurality of second electrical contacts and a plurality of electrical wires, each second electrical contact corresponding to one of the first electrical contacts and one of the plurality of electrical wires; and the unit connector assembly further comprises a plurality of third electrical contacts, each corresponding to one of the electrical wires and one of a plurality of fourth electrical contacts of at least one of the first unit and the second unit;
electrical communication according to some embodiments comprising at least one of power, control and data;
a conduit capable of housing the at least one electrical wire, the conduit is integral with fluid delivery tube;
at least one electrical contact comprises a conductive pin which is received in a recess for flaking electrical connection with another electrical contact;
monitoring means for limiting the usage of one or more components of the apparatus or system and/or the apparatus or system as a whole, which may be based on a number of uses of the component and/or apparatus or system within a predetermined period of time, based on a number of uses of the component and/or apparatus or system is for a limited time period since first usage—which may be a period of 3 days;
the fluid unit comprises a fluid dispensing device;
the treatment element includes means for applying at least one of the treatments selected from the list consisting of: heating, cooling, intermittent temperature change, temperature stabilization, mechanical vibration, acoustic vibration, massaging, ultrasound, suction, electric current, magnetic field, electric field, optical energy, radio frequency irradiation, and microwave irradiation;
identification means to provide a unique identifier for one or more components of the apparatus or system, the apparatus or system as a whole, and a package for any of the foregoing, where the identification means may include an optical bar code and/or an electronic identification means including a Maxim DS2433 1-Wire EEPROM or memory element or RFID tag;
means for electronically reading the electronic identification means;
electronic calibration means to provide calibration for a component of the apparatus or system, and/or to provide calibration for the apparatus or system as a whole, where the calibration means may be electronic calibration means comprising a Maxim DS2433 1-Wire EEPROM or memory element or RFID tag;
the second unit includes means for electronically reading the electronic calibration means;
a processor to at least one of operate, control, read, measure, collect data and process data;
a sensor selected from the group consisting of: temperature, body analyte, motion, radiation, and RF;
the catheter is selected from the group consisting of a single lumen catheter, double lumen catheter and multi-lumen catheter;
the treatment element further comprises an electromagnetic unit, which may generate an electric field, a magnetic field, light irradiation, radiofrequency (“RF”) irradiation and microwave (“MW”) irradiation or a combination thereof;
the treatment element further comprises a heating and/or cooling unit;
the treatment element further comprises a vibration unit selected from the group consisting of massaging, mechanical vibrations, acoustic vibrations and ultrasound;
the treatment element further comprises a suction unit and/or pressurization unit;
the treatment element comprises a substance delivery device for delivering an additional substance or chemical to said region;

In some embodiments of the invention, a method of delivering therapeutic fluid to a patient is provided that may include providing an infusion set according to all or a portion of any of such infusion (or other) embodiments disclosed in the present application, transcutaneously inserting the catheter into the patient, the insertion being either with the catheter alone, or as part of the catheter assembly, securing at least one of the treatment element, catheter assembly and catheter connector assembly to the patient, connecting the catheter connector assembly to the catheter, wherein such connection can occur either before or after transcutaneous insertion of the catheter, securing the unit connector assembly to at least the fluid unit, enabling the fluid unit to deliver therapeutic fluid to the patient via the catheter, applying treatment via the treatment element either before, during and/or after delivering the therapeutic fluid, optionally monitoring at least one of a body analyte, temperature, fluid unit activation and optionally applying the treatment based on the monitoring.

In some embodiments of the present invention, a method of delivering therapeutic fluid to a patient may include providing an infusion set for infusing therapeutic fluid to a patient, where the infusion set may comprise all or a portion of any of the infusion set embodiments (or other embodiments) disclosed in the present application, which may include a monitoring means for limiting the usage of the infusion set. The method may also include identifying the infusion set by a control unit, calculating the usage period of said infusion set by control unit and stopping the infusion set usage once the usage period limitation reached.

Such method embodiments may also include:
a usage limitation of a time period of 3 days;
providing identification means to provide a unique identifier for one or more components and/or its package and/or the provided system as a whole, where the identification means may comprise electronic identification means to provide a unique electronic identifier for one or more components and/or the provided system as a whole, including a Maxim DS2433 1-Wire EEPROM or memory element or RFID tag; alternatively, the identification means may be an optical bar code;

These and other embodiments, objects and advantages of the present invention will be even more clear with reference to the following detailed description and associated figures, a brief description of which is provided below.

### BRIEF DESCRIPTION OF THE DRAWINGS

FIG. 1 illustrates a top view of an exemplary detachable part of an infusion set connector that includes electrical connection, according to some embodiments of the present invention.

FIG. 2 illustrates a perspective wire-frame view of an exemplary detachable part of an infusion set connector that includes electrical connection, according to some embodiments of the present invention.

FIG. 3 illustrates a cross-sectional view of an exemplary infusion set connector that includes electrical connection, according to some embodiments of the present invention.

FIG. 4A illustrates an exploded perspective view of components of an exemplary catheter part of an infusion set that combines a heating element, according to some embodiments of the present invention.

FIG. 4B illustrates a bottom-perspective view of component 22 as shown in FIG. 4A, according to some embodiments of the present invention.

FIG. 5A illustrates a top perspective view of an exemplary cover portion of a catheter part of an infusion set which is movable with respect to other components/portions of the catheter part, and is shown in a closed position, covering an electrical connection area, according to some embodiments of the present invention.

FIG. 5B illustrates a top perspective view of an exemplary cover portion of a catheter part of an infusion set which is movable with respect to other components/portions of the catheter part, and is shown in an open position revealing an electrical connection area, according to some embodiments of the present invention.

FIG. 6A illustrates perspective top view of an exemplary connection for a heating element heater for an infusion set, according to some embodiments of the present invention.

FIG. 6B illustrates enlarged perspective view of an exemplary connection for a heating element for an infusion set, according to some embodiments of the present invention.

FIG. 7 illustrates a top perspective view of an exemplary infusion set that includes electrical connection, according to some embodiments of the present invention.

FIG. 8 illustrates a top exploded perspective view of the exemplary infusion set of FIG. 7 in disconnection, according to some embodiments of the present invention.

FIG. 9 illustrates a bottom exploded view of an exemplary infusion set of FIGS. 7-8, according to some embodiments of the present invention.

FIG. 10 illustrates a top perspective view of an exemplary infusion set connector that includes electrical connection, according to some embodiments of the present invention.

FIG. 11 illustrates a top exploded view of the exemplary infusion set connector of FIG. 10, according to some embodiments of the present invention.

FIG. 12A illustrates an exemplary infusion set and pump connection, utilizing a catheter pump-side connector that includes electrical connection, according to some embodiments of the present invention.

FIG. 12B illustrates an exemplary infusion set pump-side connector that includes electrical connection, according to some embodiments of the present invention.

FIG. 12C illustrates an exemplary pump-side connection for connecting with the connector of the infusion set of FIG. 12B (assembled connection shown in FIG. 12A), according to some embodiments of the present invention.

FIG. 13A illustrates an exemplary infusion set and pump connection, utilizing a two-component, catheter pump-side connector that includes electrical connection, according to some embodiments of the present invention.

FIG. 13B illustrates an exemplary infusion set pump-side connector, utilizing a two-component connector that includes electrical connection, according to some embodiments of the present invention.

FIG. 13C illustrates an exemplary pump-side, two-component connection for connecting with the connector of the infusion set of FIG. 13B (assembled connection shown in FIG. 13A), according to some embodiments of the present invention.

FIG. 14A illustrates an assembled pump and infusion set connection, including a housing for containing power and other various components (e.g., sensor(s)) for a treatment element included with the infusion set, the connector including electrical connection, according to some embodiments of the present invention.

FIG. 14B illustrates the infusion set, pump-side connector and housing, as also shown in FIG. 14A, the connector including electrical connection, according to some embodiments of the present invention.

FIG. 15 illustrates an assembled pump and infusion set connection, including a housing for containing power and other various components (e.g., sensor(s)) for a treatment element included with the infusion set, the connector including electrical connection, according to some embodiments of the present invention.

FIG. 16 illustrates an exemplary infusion set pump-side connector that includes electrical connection, according to some embodiments of the present invention.

### DETAILED DESCRIPTION OF THE INVENTION

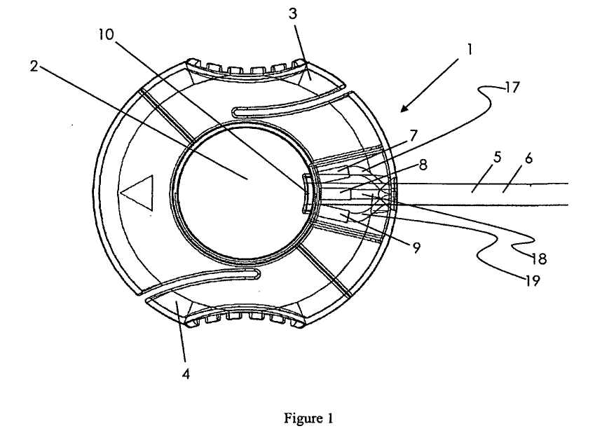

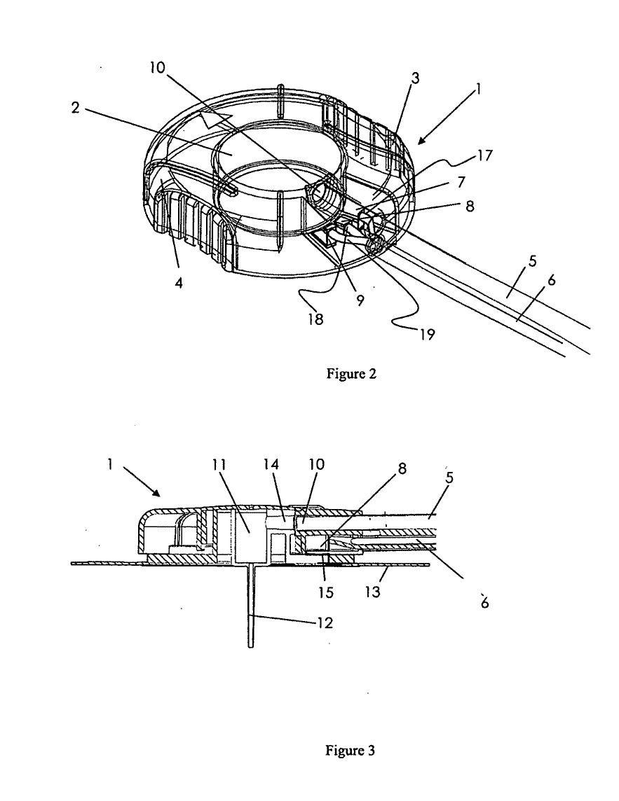

A first example of infusion set connector that connects both the catheter tube and electrical wires is shown in FIGS. 1-6, according to some embodiments of the invention. It should be noted that the same labels are used to indicate same parts in FIGS. 1-6. FIGS. 1 and 2 show the detachable part 1 (catheter connector assembly) of the connector in upper and perspective views, respectively. This part is connected to the catheter assembly part (not shown in FIGS. 1 and 2), which is inserted through the large opening 2. The detachable part 1 is then rotated by about 20° clockwise to lock the connection. The connection is locked by locking two small pins located on flexible beams 3 and 4, which are inserted into matching slits, when the detachable part 1 of the connector reaches a locking angle. The detachable part can be disconnected by pressing both beams 3 and 4 and then rotating the detachable part 1 of the connector in a counterclockwise direction to disconnect it.

In some embodiments, the detachable part 1 of the connector also includes an electrical cable 6 connected to the drug delivery pump and/or a third unit (as previously noted, the term “third unit” may also be used interchangeably with “second unit” and/or auxiliary unit”), as disclosed in the Commonly Owned Applications. Cable 6 may be attached to the drug delivery tube 5 along the entire length of tube 5, if connected to the drug delivery pump (not shown in FIGS. 1 and 2). In some embodiments, cable 6 may be attached along a portion of the length of tube 5, if connected to a third unit (not shown in FIGS. 1 and 2). The third unit may be attached to the drug delivery pump and/or may only be partially attached and/or not attached at all. The electrical cable 6 may provide power and/or communication and/or control to the treatment element and may enable reading of sensors to monitor the treatment level and/or treatment effect on the tissue and/or additional physiological parameters of the drug infused tissue vicinity and/or the skin.

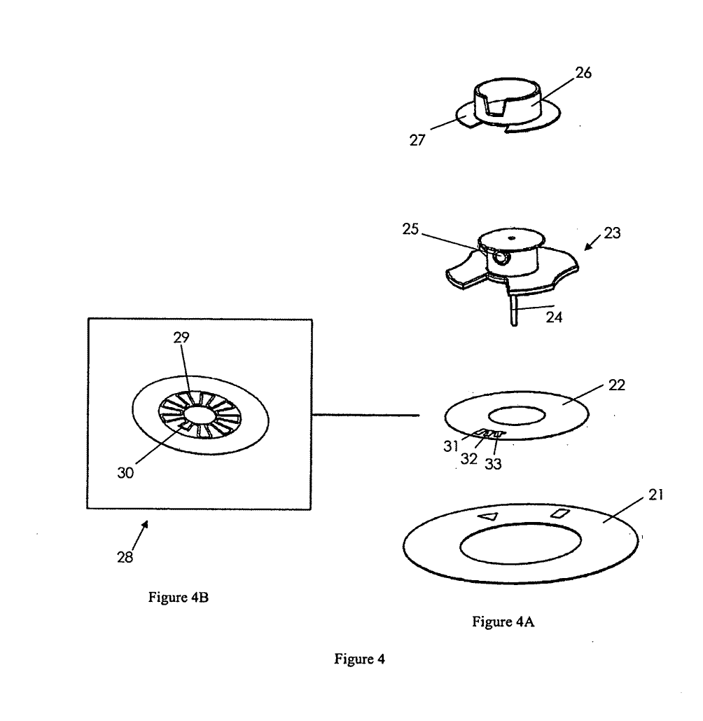

As illustrated in FIGS. 1-6, the electrical cable 6 may include three wires ending with three corresponding electrical contacts 7-9 that electrically connect the detachable part of the connector to the catheter part. The three wires 17-19 may be used for providing power to the treatment device, such as the heater shown in FIG. 4, and/or for reading at least one temperature sensor to regulate temperature of the skin at the desired temperature, as disclosed in the Commonly Owned Applications. In some embodiments, one of the three wires 17 may serve as a common ground for both the treatment element and to the at least one sensor. In some embodiments, a second wire 18 may provide power to the treatment element, such as the heater in the above example. The power to the heater can be switched on and off or regulated to provide the desired temperature profile for optimal treatment on one hand and to keep the patient safety on the other hand. In some embodiments, a third wire 19 may be used for reading the temperature of a single thermistor by reading the resistance between the ground wire and the third wire by the control unit located at the drug delivery pump or third unit or other options, as disclosed in the Commonly Owned Applications. In some embodiments, the third wire 19 may be used for reading the one or more digital temperature sensors, such as the temperature sensor DS18520 of Maxim, that provides the temperature reading as digital information on the same the third wire and using the common ground wire as ground. In some embodiments, additional wires are used for reading information from additional sensors, as disclosed in the Commonly Owned Applications.

The catheter part 11 of the connector 1 is shown in FIG. 3. FIG. 3 further illustrates a vertical cross-sectional view cut through the middle of the two parts of the connector. The catheter part 11 of the connector includes the catheter tube 12 and an adhesive layer 13 that can be adhered to the skin around the insertion point to secure the catheter to the skin (not shown in FIG. 3). When the detachable part 1 is locked, the opening 10 of the drug delivery tube 5 is preferably aligned with and may be located directly in front of the catheter opening 14 allowing the fluid to flow through the delivery tube 5 and through the opening 10 to the catheter 12, as shown in FIG. 3. In some embodiments when the detachable part 1 is in its locked angle, the three electrical contacts 7-9 (not shown in FIG. 3) located in detachable part 1 are preferably aligned with the three electrical contacts at the catheter part 11 of the connector. FIG. 3 illustrates this state in which the middle electrical contact 8 at the detachable part 1 is located on the middle electrical contact 15 at the catheter part 11 of the connector (other contacts are not shown). The locking mechanism described before or another locking mechanism ensure that the electrical connection between the two parts will be reliable and will not be disconnected accidentally, such as in case of pulling the drug delivery tube 5 or the electrical cable 6 or rapid movements of the user.

The catheter portion connecting to detachable portion 1, as shown in FIGS. 1-3 in an exemplary embodiment of the present invention is shown in FIGS. 4A-5B. FIG. 4A illustrates schematically an exploded view of the major components of the catheter part separated from each other and spread vertically for the purpose of illustration only. The bottom part is a circular flexible adhesive layer 21 that secures the catheter to the skin (not shown in FIG. 4A). Inside, there is a circular heating element 22 covered with an adhesive layer 21 underneath. The heating element 22 and the bottom side of the adhesive layer can be covered with a laminate (not shown in FIG. 4A), that can be peeled off by the user before insertion of the catheter. On top of the laminate, the main body 23 (e.g., catheter housing of catheter assembly) of the catheter unit is located, which includes the catheter tube 24 and the tube opening 25, attached to the detachable part of the connector described before as opening 14 illustrated in FIG. 3. On the main body 23, a ring shaped cover 26 covers the tube opening 25 and the electrical contacts 31-33 of the heater 22 when the detachable part of the connector is disconnected.

FIG. 4B depicts the underside side 28 of heating element 22. Underside 28 is covered by a patterned heating element 29 which may be in the form of a wire or resistor and includes a temperature sensor 30, and may be arranged in any arrangement, to provide heat to the area in which the element is placed. In some embodiments, the patterned heating element 29 can be manufactured by printing technology or by Printed Circuits Boards (“PCB”) manufacturing technologies as is known and accepted in the art.

In some embodiments, when the detachable part 1 of the connector is disconnected, a special “dummy” cover (non functional cover, not shown) that is designed to have similar shape and/or footprint to the detachable part can be manually attached to the catheter part instead and covers the catheter tube opening and the electrical contacts so they can be protected, for example, from dirt, infections or water, as for taking a shower. This dummy cover can lock the tube and the electrical wires. When the detachable part of the connector is to be connected the special cover is manually removed from the catheter part and the detachable part is connected instead. The special dummy cover is then retained for future use.

In some embodiments, when the detachable part 1 of the connector is disconnected, it automatically covers the connector's electrical contacts and tube opening, as shown in FIGS. 5A-B. FIGS. 5A-B show the assembled view of the parts shown in FIGS. 4A and 4B. This cover may be necessary for example when taking a shower, as mentioned before. FIG. 5A depicts the disconnected state 41 and FIG. 5B depicts the connected state 42, while the detachable portion 1 illustrated in FIGS. 1-3 is not shown. The catheter part includes, similar to the description of FIGS. 4A-B, the heater and adhesive layer 43 (label 21 in FIGS. 4A-B) and the main body 44 (label 23 of FIG. 4A). FIG. 5B depicts the open state of the electrical contacts 47 and catheter opening 48 when the connector is in the connected position 42 achieved with the twistable ring cover 46. When the detachable part (not shown in FIGS. 5A-B) is in the connected position 42 the ring shaped cover 46 (label 26 of FIG. 4A) does not cover the electrical contacts 47 (label 31-33 of FIG. 4A) and the catheter tube opening 48 (label 25 of FIG. 4A).

Conversely, FIG. 5A depicts the closed state of the electrical contacts 47 and catheter opening 48 when the connector is detached in the disconnected position 41 achieved when twistable ring cover 46 shields the contacts 47 and catheter opening 48. When the connector (not shown in FIGS. 5A-B) is disconnected the detachable part (not shown in FIGS. 5A-B) is rotated counterclockwise, as disclosed above, and rotates the ring shaped cover 45 (labeled 26 in FIG. 4A) by approximately 20° (for example), such that it covers the electrical contacts 47 and the catheter tube opening 48, as shown at the disconnected state 41. Following the 20° turn, the connector (not shown in FIG. 4A) is lifted off and disconnected. In this state, both the catheter tube opening 48 and the electrical contacts 47 are protected. When the detachable part (not shown in FIG. 4A) of the connector is reconnected or connected for the first time, that is going from closed position 41 to open position 42, it placed on the around the main body 44 and then rotated clockwise till it gets to the locking position. When the detachable part (not shown) is rotated, it rotates also the ring shaped cover 45 (label 26 of FIG. 4A) to its opened position 46 and enables contact between the tubes and the wires at both sides of the connector.

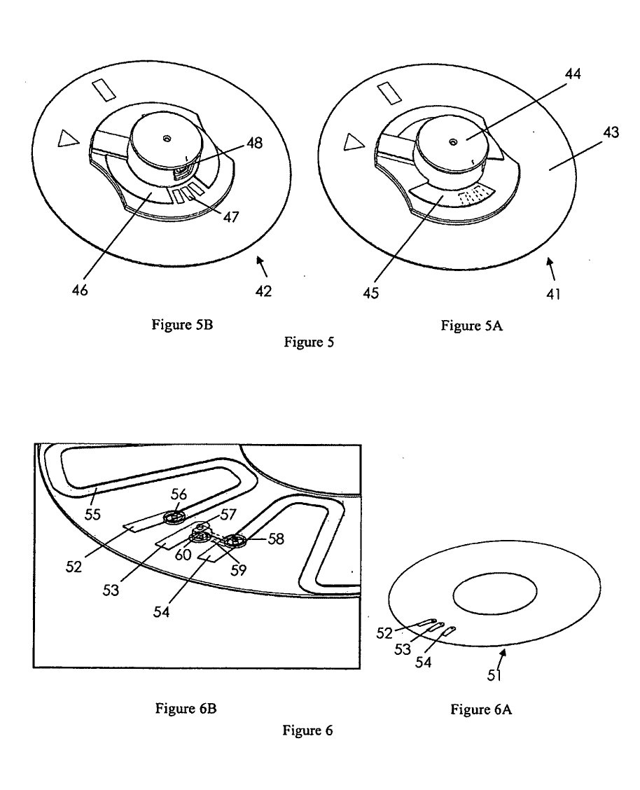

In some embodiments, the electric contacts of the treatment element, such as the heater in the current example, are disposed on the upper side of the treatment element, as shown in FIGS. 4A-B and enlarged in FIG. 6A. The heater is shown in FIG. 6A from its upper side as solid assembly 51. FIG. 6B depicts an enlarged view 55 of contacts 52-54 of FIG. 6A and shows a transparent view. As discussed above, in some embodiments, the three electrical contacts 52-54 (e.g., one or more, and preferably, multiple contacts) are disposed on the upper side of the heater assembly 51. The heater assembly 51 may be made of polyester or other known materials as is known and accepted in the art polymers or other materials or few layers of polymers that can provide the required durability and fit mass production manufacturing methods such as printing of the conductors or using printed circuit board manufacturing technologies known in the art.

In some embodiments, the polymer substrate is printed or covered with conductors on both sides as shown in FIG. 6B. The connection between the electrical contacts 52-54 to the heater itself 55 and to the temperature sensor 60 and its electrical conductor 59, all of which are printed or disposed on the bottom part of the heater, can be made through corresponding holes 56, 57 and 58, which can be coated on their inner side with an electrically conductive layer. The methods for manufacturing such conductive holes, are well known in the art and in the practice of PCB manufacturing technologies, enable connection of the conductive patterns on both sides of the heaters at low cost mass production manufacturing. Using the same flexible or rigid PCB, both for the treatment such as heating and for providing the electrical contacts of the catheter part of the connector simplifies the manufacturing process of the catheter part combined with the treatment element and reduces its cost.

In the example shown in FIG. 6B, electrical contact 54 is the common ground. Electrical contacts 52 and 54 are used for applying current to the heater conductor 55. Electrical contacts 53 and 54 are used for measuring the temperature using sensor 60, which is connected to the ground with a conductive line 59 on the bottom side of the heater. The bottom side of the heater 55 can be further coated with electrical non conducting layer, which can be thermally conductive, that protects the heater conductor 55 and the temperature sensor 60 and/or the patient skin. In some embodiments, the temperature sensor 60 is also printed on the heater polymer substance, for instance by printing a temperature sensitive low conductivity conductor. In this case, the temperature can be measured by measuring the resistance between electrical contacts 53 and 54 and applying a calibration function to get the temperature and use it to regulate the heater operation. In some embodiments, the polymer substrate of the heater includes an embedded metallic layer, such as cooper, to improve the heat conductance and the temperature uniformity across the heater.

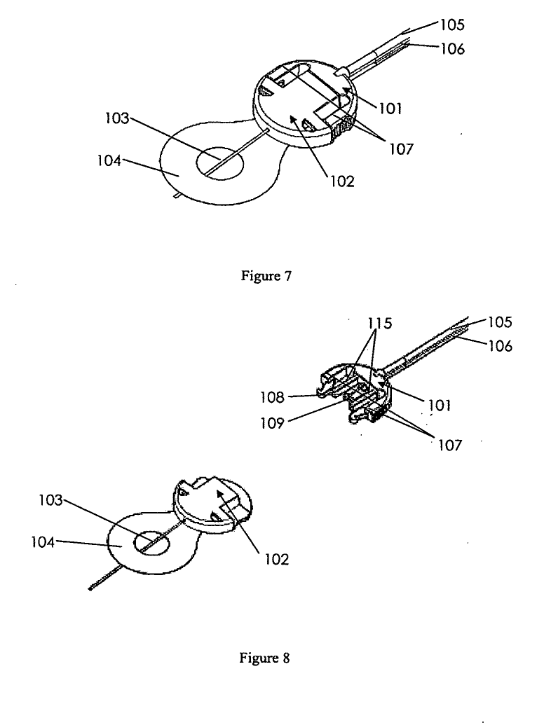

A second example of infusion set connector that connects both the catheter tube and electrical wires is shown in FIGS. 7-9. This example is based on the snap type of infusion sets, which is common for insulin delivery. FIG. 7 illustrates the two parts of the connector, the detachable part 101 and the catheter part 102, in the connected state.

The detachable part 101 of the connector can be connected to the catheter part 102 using the two flexible clips 107, which are inserted to matching slits (not shown in FIG. 7) in the catheter part 102 and which lock the connector to prevent leakage. The detachable part 101 is disconnected from the catheter part 102 by pressing both flexible clips 107 and pulling the detachable part backwards. In some embodiments, the detachable part of the connector 101 includes also an electrical cable 106 connected to the drug delivery pump and/or a third unit, as discussed above. Cable 106 may be attached to the drug delivery tube 105, as discussed above. In some embodiments, the catheter can be configured as double (i.e., multiple) lumen tube, wherein one of the lumens is used for the drug delivery and the electrical wires are disposed in the second lumen. In the example shown in FIGS. 7-9, electrical cable 106 includes three wires ending with three electrical contacts that electrically connect the detachable part of the connector to the catheter part. The catheter part of the connector includes a flexible catheter tube 103 may be inserted into the skin to the subcutaneous tissue and a flexible treatment element 104 covered by an adhesive layer adhered to the skin around the insertion point to secure the catheter to the skin.

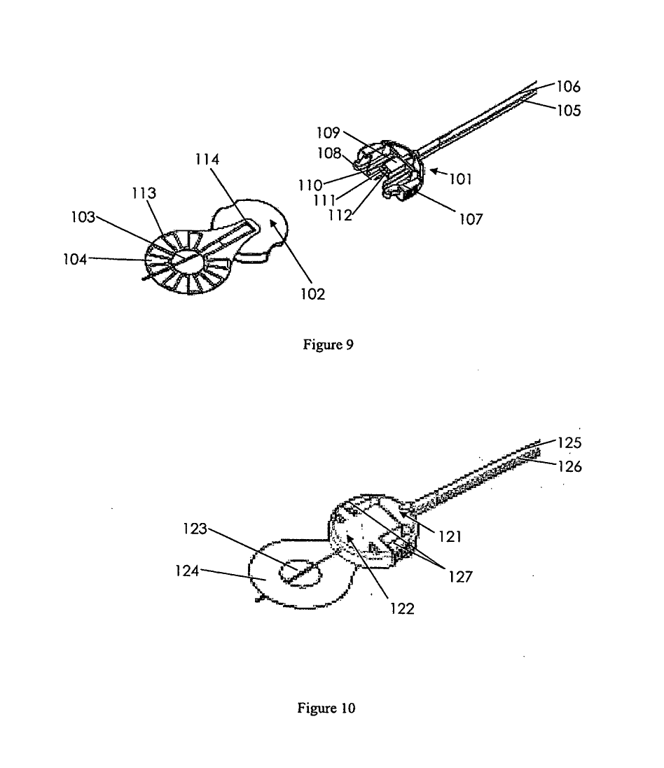

FIG. 8 illustrates upper view of the two parts of the connector in disconnected state: the detachable part 101 and the catheter part 102. In the illustrated example, the detachable part of the connector 101 includes a metallic tube 108, connected to the drug delivery tube 105 and inserted into the catheter tube in the catheter part of the connector. In the illustrated example, the detachable part of the connector 101 includes also two supporting beams 115 that fit matching holes in the catheter part of the connector 102 and provide accuracy to the connection and strength. In some embodiments, the detachable part 101 includes also an extension 109 with electrical contacts that fits a matching slit (not shown FIG. 8) in the catheter part of the connector 102. The electrical contacts 110-112 can be seen in the bottom view of the two parts of the connector in disconnected state, as illustrated in FIG. 9 in the detachable part 101. The electrical contacts on the catheter part 102 matching electrical contacts 110-112 can not be seen in this view. Those contacts on the catheter part 102 are connected to the treatment element, for example as the heater 113 and temperature sensor 114, as discussed above.

In some embodiments, when the detachable part 101 of the connector is disconnected, a special “dummy” cover (not shown in FIG. 9), which is designed similarly to the detachable part 101 and fits the catheter part 102 but without the tube 105 and the electrical cable 106, is manually attached to the catheter part instead and covers the catheter tube opening and the electrical contacts so they can be protected from dirt, infections or water, as in taking a shower. When the detachable part of the connector 101 should be connected to the special dummy cover (not shown in FIG. 9) is manually removed from the catheter part 102 and detachable part 101 is connected instead. The special cover should be kept for next time it is needed. In some embodiments not illustrated in the present application when the detachable part of the connector 101 is disconnected a special cover looks like a small door that is pushed by a spring and automatically closes the slit in the catheter part of the connector 102 when extension 109 is pulled out of this slit/socket (not shown in the Figures). When detachable part 101 is connected again to catheter part 102 extension 109 push the small door and gets into matching slit in catheter part till it gets to locked position, where contacts 110-112 on the extension 109 provides electrical contact to the matching contacts on the catheter part 102. Similarly, in some embodiments, where the detachable part of the connector 101 is disconnected and metallic tube 108 is pulled out of the catheter part 102 a special rubber “O” ring (e.g., a small circular ring) seals the catheter opening in the catheter part 102. When detachable part 101 is connected again to catheter part 102 metallic tube 108 is inserted through the O-ring and enable drug flow from the drug delivery pump through detachable part 102 to the catheter 103 in catheter unit 102 without leakage of fluids.

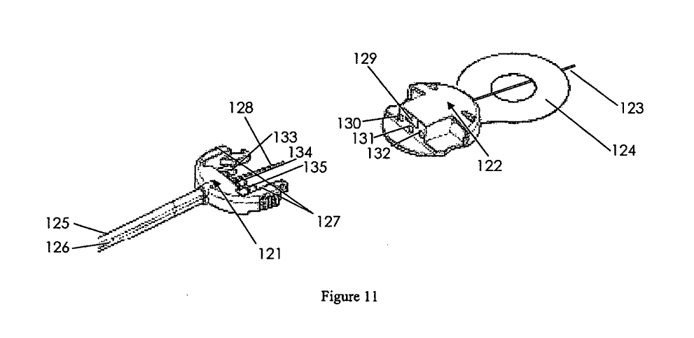

A third example of infusion set connector that connects both the catheter tube and electrical wires is shown in FIGS. 10-11. This example is based again on snap type of infusion sets, which is common for insulin delivery. FIG. 10 illustrates the two parts of the connector the detachable part 121 and the catheter part 122 in connected state.

The detachable part 121 of the connector is connected to the catheter part 122 using the two flexible clips 127, which are inserted to a matching slits in the catheter part 122 and locks the connector to prevent leakage. The detachable part 121 is disconnected from the catheter part 122 by pressing both flexible clips 127 and pulling the detachable part backwards. In some embodiments, the detachable part of the connector 121 also includes an electrical cable 126 connected to the drug delivery pump and/or a third unit, as discussed above. Cable 126 may be attached to the drug delivery tube 125 partially or all the way, as discussed above. In the example shown in FIGS. 10-11, electrical cable 126 includes three wires ending with three electrical contacts that electrically connect the detachable part of the connector to the catheter part. The catheter part of the connector includes a flexible catheter tube 123 that may be inserted into the skin to the subcutaneous tissue and a flexible treatment element 124 covered by an adhesive layer adhered to the skin around the insertion point to secure the catheter to the skin.

FIG. 11 illustrates upper view of the two parts of the connector in disconnected state the detachable part 121 and the catheter part 122. In the illustrated example, the detachable part of the connector 121 includes a metallic tube 128 connected to the drug delivery tube 125 and inserted into a matching hole 129 connected the catheter tube 123 in the catheter part of the connector 122. In some embodiments, the detachable part 121 also includes electrical pins 133-135 that can fit matching holes in the catheter part of the connector 122. The electrical pins 133-135 fit three holes 130-132 in the catheter part 122 that include electrical contacts inside each hole that provide electrical contacts to pins 133-135 when the detachable part 121 is connected. The contacts inside holes 130-132 on the catheter part 122 are connected to the treatment element, such as the heater and temperature sensor discussed above.

In some embodiments when the detachable part 121 of the connector is disconnected, a special “dummy” cover, which can be designed similarly to the detachable part 121 and configured to fit the catheter part 122 without the tube 125 and the electrical cable 126, is manually attached to the catheter part instead and covers the catheter tube's opening and the electrical contacts so they can be protected from dirt, infections or water (e.g., when taking a shower). When the detachable part of the connector 121 is connected, the special cover is manually removed from the catheter part 122 and detachable part 121 is connected instead. The special cover should be kept for next time it is needed. In some embodiments, when the detachable part of the connector 121 is disconnected a special cover looks like a small door that is pushed by a spring and automatically closes all holes 129-132 in the catheter part of the connector 122 when the metallic tube 128 and pins 133-135 are pulled out of this slit/socket 129-132. When detachable part 121 is connected again to catheter part 122 metallic tube 128 and pins 133-135 push the small door and gets into matching holes 129-132 in catheter part till it gets to locked position, where pins 133-135 provides electrical contact to the matching contacts inside holes 130-132 on the catheter part 122. Similarly, in some embodiments, when the detachable part of the connector 121 is disconnected and metallic tube 128 is pulled out of the catheter part hole 129 a special rubber O ring seals the catheter opening in the catheter part 122. When detachable part 121 is connected again to catheter part 122 metallic tube 128 is inserted through the O ring and enable drug flow from the drug delivery pump through detachable part 122 to the catheter 102 in catheter unit 122 without leakage of fluids.

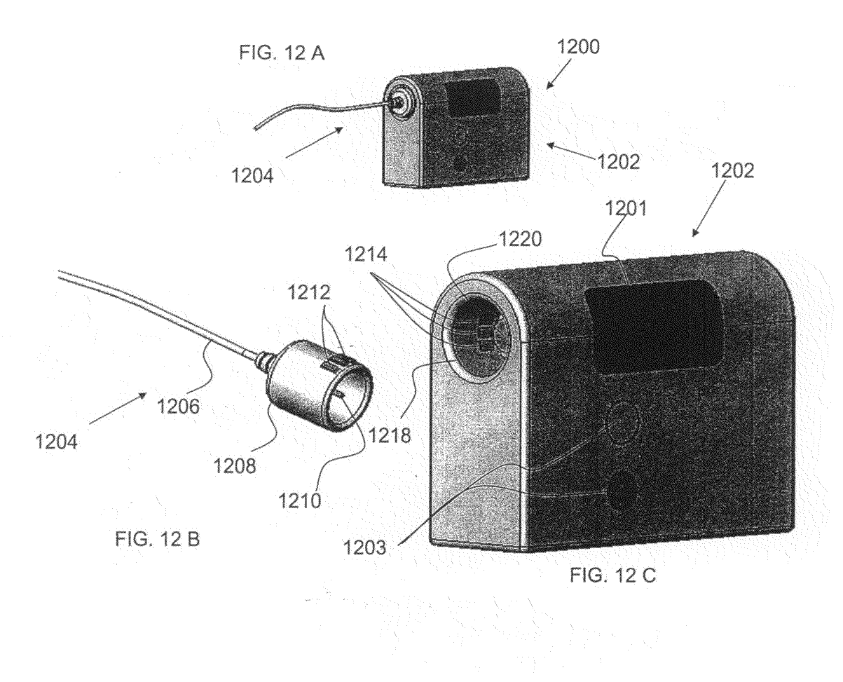

FIGS. 12A-C illustrate an optional embodiment of the present invention related to an infusion conduit having electrical leads integrated into a single connector that connects to the drug delivery pump. FIG. 12A depicts the assembled drug delivery pump 1200 having the pump 1202 and connector 1204 attached thereto.

FIG. 12B depicts a close up view of the connector 1204 removed from the drug delivery pump 1200. In some embodiments, the connector 1204 includes a catheter 1206 with electrical wires, as discussed above, a male connector housing 1208, a needle 1210 and electrical leads 1212. Connector housing 1208 includes a needle 1210 and the plurality of electrical leads 1212. In some embodiments, the housing 1208 is a male connector that fits into its matching female connector placed in the delivery pump 1202, as shown in FIG. 12A. In some embodiments, the needle 1210 extracts the drug to be delivered from the drug storage compartment integrated into the delivery pump 1202 shown in FIG. 12A. In some embodiments, the drug to be delivered is then conveyed from needle 1202 to catheter 1206 to make its way to the targeted drug delivery site. A plurality of electrical leads 1212 are located on an external face, along the perimeter of the connector housing 1208. The electrical leads 1212 are configured to be contact leads that have corresponding contact leads at the delivery pump 1202 in order to close the power supply loop. The electrical leads 1212 are used to provide a power supply for the tissue treatment element (not shown in FIGS. 12A-C) and are configured to read optional sensors, as discussed in the Commonly Owned Applications. The electrical current may be delivered forward via the catheter 1206 that may have an integrated electrical conducting member (not shown in FIGS. 12A-C). The catheter itself can provide the requisite power supply while controlling the tissue treatment element and delivering the drug to the targeted tissue. In some embodiments, for the ease of alignment, the male connector housing 1208 can have two pins (not shown in FIGS. 12A-C), while the female recess 1218 has two corresponding small spiraled slits (not shown in FIGS. 12A-C) that are configured to lock-in with the male connector housing 1208 in position, thus, insuring the proper alignment of connectors 1208 and 1218.

FIG. 12C is more detailed view of the drug delivery pump 1202 shown in FIG. 12A. Delivery pump 1202 includes the female connector recess 1218 utilized to accept the male connector 1208, as shown in FIG. 12B. In some embodiments, the female connector recess 1218 includes a drug compartment membrane 1220 that corresponds to the needle 1210 allowing the extraction of the drug to drug delivery catheter 1206. The female connector recess 1218 further includes a plurality of electrical contact leads 1214 that are located along the inner surface of recess 1218. The electrical contracts 1218 are configured to align with the corresponding electrical contact leads 1214 when the contact leads 1214 and 1218 form a close electrical circuit, thus, allowing the delivery of electrical current via the catheter 1206. The pump 1202 may also include a display 1201 and at least one or more indicators 1203. The indicators 1203 may be in the form of an LED or the like. Indicators' 1203 functions may include, but are not limited to, power indication, battery status indication, error indication or any other desired indication.

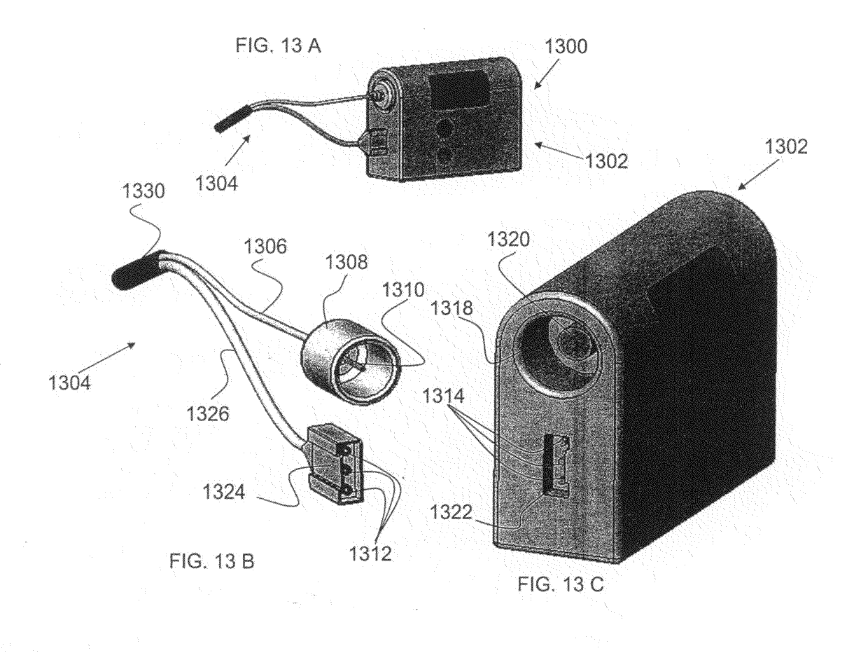

FIG. 13A-C illustrate an exemplary infusion pipe having electrical leads integrated into a single connector that connects to the drug delivery pump, according to some embodiments of the present invention. FIG. 13A depicts the assembled drug delivery pump 1300 having a pump 1302 and a connector 1304 attached thereto.

FIG. 13B depicts a close up view of connector 1304 removed from drug delivery pump 1304. In some embodiments, the connector assembly 1304 includes a catheter 1306, a male drug delivery connector housing 1308, a needle 1310, an electrical lead wire 1326, male electrical contact leads 1312, and a male power connector 1324. The drug delivery connector housing 1308 can include the needle 1310. The housing 1308 is a male connector that is configured to fit into its matching female connector when placed in the delivery pump 1302, as shown in FIG. 13A. The needle 1310 can be configured to extract the drug to be delivered from the drug storage compartment integrated into the delivery pump 1302 of FIG. 13A. The drug to be delivered is then conveyed from the needle 1302 to the catheter 1306 to make its way to the targeted drug delivery site. The catheter 1306 is joined with electrical lead wire 1326 via tube connector 1330.

The electrical lead wire 1326 conducts power to tissue treatment elements (not shown in FIGS. 13A-C) of the drug delivery device. The power may be obtained from the drug delivery pump 1302. The lead wire 1326 is connected via male power connector 1324. The male power connector 1324 is utilized to close the loop with its corresponding female power connector located on the pump 1302. The male connector 1324 and its female counterpart are specifically shaped to ensure proper alignment and connection. The male connector 1324 include a plurality of female electrical leads 1312 encased within the male connector 1324. The female electrical leads 1312 can be contact leads that have corresponding male contract leads at delivery pump 1302 that allow the power supply circuit to be closed.

FIG. 13C depicts in greater details the drug delivery pump 1302 shown in FIG. 13A. The delivery pump 1302 includes a female connector recess 1318 utilized to accept male connector 1308, as shown in FIG. 13B. The female connector recess 1318 includes a drug compartment seal 1320 for example including but not limited to a membrane or an O-shaped ring seal or the like, that corresponds to the needle 1310 allowing the extraction of the drug to the drug delivery catheter 1306.

The drug delivery pump 1302 further includes a female power connector recess 1322 that has a plurality of male electrical contact leads 1314. The female power connector recess 1322 corresponds to the shape of male power connector 1324 to allow unidirectional attachment ensuring that male contact leads 1314 are in contact with female contact leads 1312. The contact leads 1314 and 1312 close an electrical circuit allowing sensor reading and/or delivery of electrical current via power supply wire 1326 that delivers power to a tissue treat element (not shown in FIGS. 13A-C) as part of the drug delivery device. The connector 1324 can also be connected also to a third or an auxiliary unit, as discussed in the Commonly Owned Applications. The auxiliary unit may, for example, include the drug delivery housing, a bag, or an appendage to the drug delivery pump, or the like.

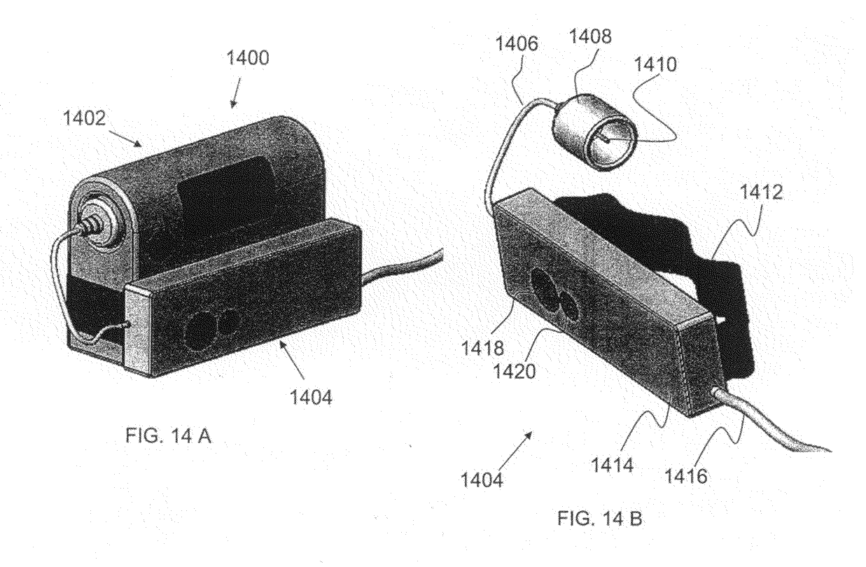

FIGS. 14A and 14B illustrate an exemplary embodiment of the present invention wherein power for the tissue treatment element (not shown in FIGS. 14A-B) is not derived from the delivery pump 1402, as shown in FIGS. 12A-C and 13A-C. Rather, an independent third unit can be employed that provides the power source and control for the treatment element (not shown in FIGS. 14A-B). FIG. 14A depicts the drug delivery apparatus 1400 having a drug delivery pump 1402 and a third unit 1404. The third unit 1404 provides power for a tissue treatment element (not shown in FIGS. 14A-B) used as a part of the drug delivery device ensemble. The third unit 1404 also provides control for any sensors of the tissue treatment or any other sensors, as discussed in the Commonly Owned Applications. FIG. 14B depicts the third unit 1404 in greater detail. The third unit 1404 connects to the drug delivery pump 1402 to extract the drug that is to be delivered. As shown in FIGS. 12A-C and 13A-C, the pump 1402 (similar to the pumps 1202 and 1302) connects and delivers a drug from its intrinsic drug compartment (not shown in FIGS. 14A-B) to the third unit 1414 using a needle 1410. The needle 1410 is enclosed within a male drug delivery connector housing 1408 allowing the drug to pass to the drug delivery catheter 1406. Catheter 1406 enters third unit 1414 and passes through to catheter 1416 that continues to the drug delivery site (not shown in FIGS. 14A-B). The catheter 1416 can also include an electrical conducting wire to deliver electrical power and to control a tissue treatment element at or near the tissue target site (not shown in FIGS. 14A-B).

The third unit 1414 can be coupled to the drug pump 1402 with a strap 1412. The strap 1412 may be an elastic strap that may easily be adjusted to fit over the pump 1402. The strap 1412 can include a hook-and-latch assembly, such as Velcro® or the like, coupling mechanism to comfortably couple third unit 1414 to pump 1402. The third unit 1414 can be attached to the drug delivery pump 1402 with a clip or an adaptor to securely couple the third unit 1414 to the pump 1402. The third unit 1414 can have an internal power source that is portable. The power source can be, but is not limited to, an alkaline battery, a lithium battery, a rechargeable battery or any other portable power source configured to generate electrical power to be conveyed to the tissue treatment element (not shown in FIGS. 14A-B). The third unit 1414 can also include a solenoid or other types of sensors (not shown in FIGS. 14A-B) to detect when pump 1402 is actively pumping or delivering the drug through to the catheter 1406, to utilize the activity to generate and initiate electrical power for tissue treatment element at the tissue target site (not shown in FIGS. 14A-B). The third unit 1414 may be controllably activated or deactivated using a button 1418, while indicator 1420 may communicate the status of the power supply, functioning status of the treatment element, or system errors or the like. The indicator 1420 may be in the form of an LED or any other type indicator.

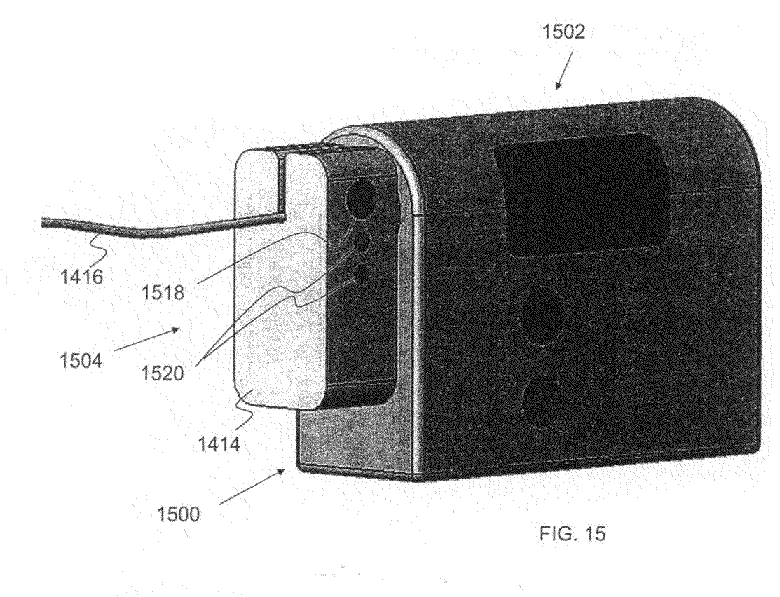

FIG. 15 illustrates another exemplary embodiment of the third unit 1414 shown in FIG. 14B where the coupling means of the drug delivery pump 1502 is different and does not utilize a strap. An assembly 1500 includes a drug delivery pump 1502 and a third unit 1504. The third unit 1504 can be coupled to the pump 1502 directly via its pump drug delivery connectors (not shown in FIG. 15, but discussed in connection with FIGS. 14A-B, for example, wherein the female drug connector recess 1318 is firmly connected to the male drug connector 1308). The function of the third unit 1504 and the catheter 1516 remain the same as 1414 and 1416. The third unit 1504 may be controllably activated or deactivated with a button 1518, while indicators 1520 communicate status of the power supply, status of the treatment element, system errors or the like. Indicators 1520 may be in the form of an LED or any other suitable indicator.

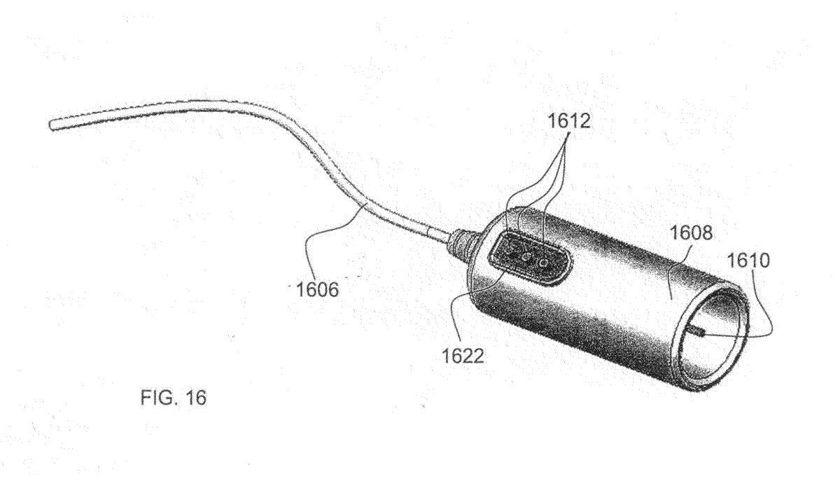

FIG. 16 depicts a connector 1600 as an alternative connector to the one shown in FIGS. 12A-C, as the connector assembly 1204. An external or third unit, for example, may be only a power source or a battery (not shown in FIG. 16), or third unit 1414 as depicted in FIG. 15 may coupled to a male connector housing 1608 via a female power connector recess 1622.

In some embodiments, connection to drug delivery pump (not shown in FIG. 16) is carried out, as shown in FIGS. 12A-C and 13A-C. The male connector housing 1608 includes a needle 1610, a female power connector recess 1622 that further internally includes a plurality of male electrical connector leads 1612. The housing 1608 can be a male connector that fits into its matching female connector placed in the delivery pump (not shown in FIG. 16, but illustrated in FIG. 12A). The needle 1610 functions to extract the drug to be delivered from the drug storage compartment integrated into the delivery pump (not shown in FIG. 16). The drug to be delivered is then conveyed from the needle 1202 to the catheter 1206 to make its way to the targeted drug delivery site. Once a power source is connected to connector recess 1622 thereby forming a closed circuit electrical, power may be provided to a tissue treatment element via catheter 1606 (not shown in FIG. 16).

Example embodiments of the methods and components of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments, but should be defined only in accordance with the following claims and their equivalents.

Any and all references to patents, patent applications, articles and other published and non-published documents made in the present disclosure are herein incorporated by reference in their entirety.

1-75. (canceled)

76. An infusion apparatus for delivering a therapeutic fluid from a fluid source to a patient comprising:
a catheter assembly including:
a catheter housing;
a catheter capable of insertion transcutaneously into a patient;
a fluid inlet port in fluid communication with the catheter; and
a treatment element capable of providing a treatment adjacent a catheter;
at least one electrical contact in electrical communication with the treatment element;
a securing element for securing catheter assembly to the patient;
a connector assembly capable of providing electrical connection and fluid communication between the catheter assembly and the fluid source, including:
a fluid delivery tube capable of communicating therapeutic fluid from the fluid source to the catheter assembly; and
at least one electrical wire in electrical communication with the at least one electrical contact.

77. The apparatus according to claim 76, wherein the connector assembly includes
a catheter connector capable of connection with the catheter assembly, the connector assembly, including
a catheter connector housing;
the fluid delivery tube capable of communicating therapeutic fluid from a fluid unit to a catheter positioned within the catheter housing upon connection of the catheter connector assembly with the catheter assembly;
at least a second electrical contact; and,
the at least one electrical wire in electrical communication with the at least second electrical contact; and,
a fluid connector capable of connection with the fluid unit, including
a fluid unit connector housing;
a fluid receiving port capable of receiving therapeutic fluid from the fluid unit upon connection of the fluid connector with the fluid unit; and,
at least a third electrical contact in communication with the at least one electrical wire capable of allowing electrical communication with at least a fourth electrical contact provided on the at least of the fluid unit and a second unit.

78. The apparatus according to claim 77, further comprising a controller that controls operation of the treatment element.

79. The apparatus according to claim 76, wherein fluid source includes an insulin pump.

80. The apparatus according to claim 77, wherein the catheter assembly includes at least one sensor.

81. The apparatus according to claim 77, wherein the electrical wires of the connector assembly are embedded into a wall of the fluid delivery tube.

82. The apparatus according to claim 81, further comprising a movable or removable cover provided with the catheter housing and capable of covering at least one of the at least one electrical contact and the fluid inlet port at least prior to use.

83. The apparatus according to claim 82, wherein the cover is manually removable, such that removing the cover exposes at least one of the first electrical contact and the fluid inlet port.

84. The apparatus according to claim 82, wherein the cover is rotatable, such that rotating the cover exposes at least one of the first electrical contact and the fluid inlet port.

85. The apparatus according to claims 82, further comprising a locking member for locking the catheter connector assembly to the catheter assembly.

86. The apparatus according to claim 76, wherein the treatment element is capable of applying at least one of the treatments selected from the group consisting of: heating, cooling, intermittent temperature change, temperature stabilization, mechanical vibration, acoustic vibration, massaging, ultrasound, suction, electric current, magnetic field, electric field, optical energy, radio frequency irradiation, and microwave irradiation.

87. The apparatus according to claim 77, further comprising:
monitoring means disposed on at least one of the fluid unit and the second unit and configured to limit usage of the infusion apparatus based on a number of uses of the infusion apparatus within a predetermined period of time beginning with a first usage of the infusion apparatus.

88. The apparatus according to claim 87, further comprising
identification means to provide a unique identifier for at least one of the components selected from a group consisting of: the catheter assembly, the catheter connector, the fluid connector, the treatment element, and packaging of at least one of the catheter assembly, the catheter connector, the fluid connector, and the treatment element;
wherein the fluid source and/or the second source include means for electronically reading the identification means.

89. The apparatus according to claim 88, wherein the identification means is selected from a group consisting of: a 1-Wire EEPROM, a memory element, a RFID tag, and an optical barcode.

90. The apparatus according to claim 77, further comprising
electronic calibration means to provide a calibration for a component of the apparatus or system.

91. The apparatus according to claim 80, wherein the sensor is at least one sensor selected from the group consisting of: a temperature sensor, bodily analyte sensor, a motion sensor, a radiation sensor, and a RF sensor.

92. A method of delivering therapeutic fluid to a patient, the method comprising the steps of:
providing an infusion set for infusing therapeutic fluid to a patient, the infusion set comprising:
a treatment element capable of providing a treatment adjacent a catheter
at least one first electrical contact;
a catheter assembly including:
a catheter housing;
a catheter capable of insertion transcutaneously into a patient, wherein the catheter is in fluid communication with the fluid inlet port;
a fluid inlet port in fluid communication with the catheter; and
a movable or removable cover capable of covering at least one of the at least one electrical contact and the fluid inlet port at least prior to use;
a catheter connector assembly capable of connection with the catheter assembly, the connector assembly including:
a catheter connector housing;
a fluid delivery tube capable of communicating therapeutic fluid from a fluid source to the catheter upon connection of the catheter connector assembly with the catheter assembly,
at least one second electrical contact, and
at least one electrical wire in electrical communication with the at least one second electrical contact;
a fluid source connector assembly capable of connection with at least the fluid source, the source connector assembly including:
a fluid source connector housing;
a fluid receiving port capable of receiving therapeutic fluid from the fluid source upon connection of the source connector assembly with the fluid source, and
at least one third electrical contact in communication with the at least one electrical wire capable of allowing electrical communication a fourth electrical contact provided on at least one of the fluid source and a second source;
and
a securing member for securing at least one of the treatment element, catheter assembly and catheter connector assembly to the patient;
transcutaneously inserting the catheter into the patient, the insertion being either with the catheter alone, or as part of the catheter assembly;
securing at least one of the treatment element, catheter assembly and catheter connector assembly to the patient;
connecting the catheter connector assembly to the catheter, wherein such connection can occur either before or after transcutaneous insertion of the catheter;
securing the source connector assembly to at least the fluid source;
enabling the fluid source to deliver therapeutic fluid to the patient via the catheter;
applying treatment via the treatment element either before, during and/or after delivering the therapeutic fluid;
optionally monitoring at least one of a body analyte, temperature, fluid source activation; and
optionally applying the treatment based on the monitoring.

93. A method of delivering therapeutic fluid to a patient, the method comprising the steps of:
providing an infusion set for infusing therapeutic fluid to a patient, the infusion set comprising:
a treatment element capable of providing a treatment adjacent a catheter;
at least one first electrical contact;
a catheter assembly including:
a catheter housing;
a catheter capable of insertion transcutaneously into a patient, wherein the catheter is in fluid communication with the fluid inlet port;
a fluid inlet port in fluid communication with the catheter; and
a catheter connector assembly capable of connection with the catheter assembly, the connector assembly including:
a catheter connector housing;
a fluid delivery tube capable of communicating therapeutic fluid from a fluid source to the catheter upon connection of the catheter connector assembly with the catheter assembly,
at least one second electrical contact, and
at least one electrical wire in electrical communication with the at least one second electrical contact;
a fluid source connector assembly capable of connection with at least the fluid source, the source connector assembly including:
a fluid source connector housing;
a fluid receiving port capable of receiving therapeutic fluid from the fluid source upon connection of the source connector assembly with the fluid source, and
at least one third electrical contact in communication with the at least one electrical wire capable of allowing electrical communication a fourth electrical contact provided on at least one of the fluid source and a second source;
and
a securing member for securing at least one of the treatment element, catheter assembly and catheter connector assembly to the patient;
transcutaneously inserting the catheter into the patient, the insertion being either with the catheter alone, or as part of the catheter assembly;
securing at least one of the treatment element, catheter assembly and catheter connector assembly to the patient;
connecting the catheter connector assembly to the catheter, wherein such connection can occur either before or after transcutaneous insertion of the catheter;
securing the source connector assembly to at least the fluid source;
enabling the fluid source to deliver therapeutic fluid to the patient via the catheter; and
applying treatment via the treatment element either before, during and/or after delivering the therapeutic fluid.

94. The method according to claim 93, wherein the infusion set includes
identification means for identifying the infusion set;
monitoring means for limiting the usage of the infusion set;
the method further comprises
using a controller, identifying the infusion set;
using the controller, calculating a usage period of the infusion set;
terminating usage of the infusion set usage when a limitation on the usage period is reached.

95. The method according to claim 94, wherein the identification means is capable of providing a unique identifier for components selected from a group consisting of: the catheter assembly, the catheter connector, the fluid source connector, the treatment element and packaging of at least one of the catheter assembly, the catheter connector assembly, the source connector assembly, and the treatment element.
